(19) United States
(12) Patent Application Publication
(10) Pub. No.: US 2005/0271746 A1
(43) Pub. Date: Dec. 8, 2005

(54) Topical treatments for abnormal biological conditions and method of topically treating such conditions

(76) Inventors: Chun Lim Abbott, Pittsburgh, PA (US); Dominic Abbott, Pittsburgh, PA (US); Meir Sacks, Pittsburgh, PA (US)

Correspondence Address: EPSTEIN & GERKEN, 1901 RESEARCH BOULEVARD, SUITE 340, ROCKVILLE, MD, 20850, US

(21) Appl. No.: 11/131,923

(22) Filed: May 18, 2005

Related U.S. Application Data
(60) Provisional application No. 60/571,893, filed on May 18, 2004.

(51) Int. Cl. A61K 33/26; A61K 33/24

(52) U.S. Cl. 424/647; 424/655

(57) ABSTRACT

A topical treatment for abnormal biological conditions occurring on an anatomical surface of a body comprises a treatment substance including chromium oxide, an iron compound and a pharmaceutically acceptable carrier in a consistency for topical application to the anatomical surface of the body. A method of treating an abnormal biological condition occurring on an anatomical surface of a body involves applying a treatment substance comprising chromium oxide, an iron compound and a pharmaceutically acceptable carrier to the site of an actual or potential abnormal biological condition on the anatomical surface of the body. Treatment involves correction of an actual abnormal biological condition or prevention of a potential abnormal biological condition.

### CROSS-REFERENCE TO RELATED PATENT APPLICATION

This application claims priority from U.S. Provisional Patent Application Ser. No. 60/571,893 filed May 18, 2004, the entire disclosure of which is incorporated herein by reference.

### BACKGROUND OF THE INVENTION

1. Field of the Invention

The present invention relates generally to topical treatments for abnormal biological conditions and, more particularly, to topical treatment substances containing metallic compositions and to methods involving the topical application of treatment substances containing metallic compositions to treat abnormal biological conditions including abnormal fungal, bacterial, biofilm, viral, inflammatory and neoplastic biological conditions.

2. Brief Discussion of the Related Art

Various abnormal biological conditions occur on anatomical surfaces of the body, and such abnormal biological conditions include abnormal fungal, bacterial, biofilm, viral, inflammatory and neoplastic biological conditions. Abnormal biological conditions may occur on virtually any anatomical surface of the body including the surfaces of anatomical cavities such as the vagina. The underlying causes of abnormal biological conditions may vary depending on the particular abnormal biological condition and may, for example, arise from internal causes such as diseases and immunity deficiencies or external causes such as burns and insect bites. The abnormal biological conditions may be symptomatic or asymptomatic. Conventional treatments for abnormal biological conditions occurring on an anatomical surface of the body include oral and topical pharmacological preparations which typically possess adverse side effects and oftentimes promote the development of drug-resistant organisms. Each type of abnormal biological condition occurring on an anatomical surface of a body may require its own specific treatment. Treatment is made more complicated where the abnormal biological condition is asymptomatic and/or difficult to conclusively diagnosis.

The treatment of abnormal biological conditions occurring on anatomical surfaces of the body exposed to fluid is made more difficult where the organisms responsible for the abnormal conditions are present as biofilm. Biofilm forms when bacteria adhere to a surface in a wet environment and begin to excrete a slimy substance that can anchor them to the surface. A biofilm may be formed by a single bacterial species, but more often includes many species of bacteria as well as fungi, algae, protozoa, debris and corrosion products. Once anchored to a surface, biofilm microorganisms may initiate a variety of detrimental reactions. Biofilms are implicated in a significant proportion of human bacterial infections.

Biofilm bacterial behavior is much more complex than planktonic or suspended cell behavior due to colonization of the biofilm organisms. Unlike suspended cells, biofilm organisms live in and populate communities. Recent studies have revealed significant differences in the level of expression of genes involved in nutrient cycling among members of a single species bacterial population exposed to the same apparent conditions. Within these populations, there appears to be a “division of labor” whereby some cells utilize available energy to turn on metabolic pathways that effect partial degradation of dead particulate matter while other cells of the same population utilize the degradation products to produce new cells. A bacterium which attaches to a surface thusly activates a different set of genes which transforms it into a significantly different organism than the same bacterium in suspension. Most bacterial studies are conducted using suspended bacterial cultures, and control strategies based on the behavior of suspended cells are less effective on biofilm cells. For example, antibiotic doses which kill suspended cells in humans may need to be increased to toxic levels in order to kill biofilm cells, and biofilms have thusly contributed to drug-resistance. Disinfection rates for biofilm cells are far below the disinfection rates for planktonic cells killed by antimicrobials.

It is evident from the foregoing that there is a need for better and more effective topical treatments for abnormal biological conditions occurring on anatomical surfaces of the body. There is also a need for non-pharmacological topical treatments for abnormal biological conditions occurring on an anatomical surface of a body. Another need exists for safer and more effective treatments for biofilms.

### SUMMARY OF THE INVENTION

The present invention pertains to topical treatments for abnormal biological conditions and to a method for topically treating abnormal biological conditions. The topical treatments according to the present invention are embodied in treatment substances of any suitable consistency for topical application on an anatomical surface of the body including the surfaces of anatomical cavities of the body. The treatment substances comprise active ingredients in any pharmaceutically acceptable carrier. The active ingredients comprise metallic compositions and may also comprise time release compounds. The metallic compositions can include a catalytic component, which may comprise hydrogen peroxide, sulfur or chlorine. The metallic compositions may comprise any metals or metal oxides, including chromium oxide, iron/sulfur oxide, iron/chloride oxide, iron chloride, and iron oxide individually or in combination.

In preferred treatment substances according to the present invention, the active ingredients have a total weight of or about 5% of the total weight of the treatment substance, with the carrier having a total weight of or about 95% of the total weight of the treatment substance. The active ingredients include chromium oxide, an iron compound, and optionally a time release compound. The iron compound includes one or more of iron oxide, iron/sulfur oxide, iron/chloride oxide or iron chloride. The chromium oxide has a weight in the range of or about 40-60% of the total weight of the active ingredients, with the remainder of the active ingredients being made up of the iron compound and, if provided, the time release compound. Where the active ingredients include a time release compound, the time release compound can have a weight in the range of or about 15-20% of the total weight of the active ingredients. The time release compound may comprise hydrogen dioxide (H₂O₂) in cream or gel form.

The iron compound can consist entirely of iron/sulfur oxide or iron oxide, particularly where the active ingredients include a time release compound. The iron compound can comprise iron/sulfur oxide in combination with either iron/chloride oxide or iron chloride. In the latter case, the iron/sulfur oxide preferably has a weight that is at least 50% of the total weight of the iron compound and, most preferably, the weight of iron/sulfur oxide is in the range of or about 50-80% of the total weight of the iron compound.

The carrier can be made in its entirety of a topical ointment or emollient such as Aquaphor® of Beiersdorf AG or Hydrophor of Geritrex Corporation. Alternatively, the carrier can be made of VanPen. The carrier can include color modifiers such as titanium dioxide.

A method of topical treatment according to the present invention involves applying the treatment substance to the site of an actual or potential abnormal biological condition on an anatomical surface of a body. The treatment substance, in the presence of oxygen, creates an oxidative chemical reaction capable of treating the actual or potential abnormal biological condition. Treatment includes normalizing the actual or existing abnormal biological condition or preventing the potential or not yet existing abnormal biological condition. A chemical reaction can be produced capable of destroying bacteria, fungi and viruses. Additionally, the reaction can be produced to have an anti-inflammatory effect. The treatment substance is capable of treating various symptomatic and asymptomatic abnormal biological conditions including abnormal fungal, bacterial, biofilm, viral, inflammatory and neoplastic biological conditions, and the treatment substance is capable of preventing, reducing or eliminating biofilm. The abnormal biological conditions treated by the present invention may be due to internal causes such as disease and immunity deficiencies or external causes such as injury, trauma, burns and insect bites. Treatment according to the present invention may be primary or preventative. The method of treatment preferably involves applying the treatment substance to the site of an actual or potential abnormal biological condition on an anatomical surface of the body at regular intervals.

Various objects and advantages of the present invention will become apparent from the following description of preferred embodiments of the invention including specific examples of treatment substances according to the present invention.

### DESCRIPTION OF THE PREFERRED EMBODIMENTS

A topical treatment according to the present invention comprises a treatment substance of suitable consistency, such as cream, gel, lotion, ointment, emollient and foam, for example, to permit topical application of the treatment substance to an anatomical surface of the body including surfaces of anatomical cavities of the body. The treatment substance comprises active ingredients in a pharmaceutically acceptable carrier. The active ingredients have a total weight of or about 5% of the total weight of the treatment substance, with the carrier having a total weight of or about 95% of the total weight of the treatment substance.

The active ingredients comprise a metallic composition and optionally a time release compound. The metallic composition comprises chromium oxide and an iron compound. The chromium oxide has a weight in the range of or about 40-60% of the total weight of the active ingredients, i.e. in the range of or about 2-3% the total weight of the treatment substance, with the remainder of the active ingredients being made up by weight of the iron compound and, if provided, the time release compound. The total weight of the iron compound is preferably in the range of or about 40-60% of the total weight of the active ingredients, i.e. in the range of or about 2-3% of the total weight of the treatment substance. Where the active ingredients include a time release compound, the weight of the time release compound is preferably in the range of or about 15-20% of the total weight of the active ingredients and, most preferably, in the range of or about 16-19% of the total weight of the active ingredients.

The iron compound includes one or more of iron oxide, iron/sulfur oxide, iron/chloride oxide or iron chloride. The iron compound can consist entirely of iron/sulfur oxide or iron oxide. Where the iron compound consists entirely of iron/sulfur oxide or iron oxide, it is preferred that the active ingredients include a time release compound. Alternatively, the iron compound can consist of iron/sulfur oxide in combination with iron/chloride oxide or iron chloride. In the latter case, the iron/sulfur oxide preferably has a weight at least about 50% the total weight of the iron compound and, most preferably, in the range of or about 50-80% of the total weight of the iron compound. Where iron/chloride oxide is a component of the iron compound, it is preferred that the weight of iron/chloride oxide be in the range of or about 20-50% the total weight of the iron compound, with the remainder of the iron compound being made up by weight of the iron/sulfur oxide. Where iron chloride is a component of the iron compound, it is preferred that the weight of iron chloride be about one third the total weight of the iron compound, with the remainder of the iron compound being made up by weight of iron/sulfur oxide.

The time release compound can be a hydrogen dioxide (H₂0₂) time release cream or gel. The carrier can be ointments such as Aquaphor® of Beiersdorf AG or Hydrophor of Geritrex Corporation, or a cream such as VanPen. The carrier can include color modifiers such as titanium dioxide.

Treatments substances according to the present invention can be formulated in accordance with the specific examples set forth below.

### EXAMPLE 1

600 g of a treatment substance is made from 30 g of active ingredients in 570 g of a carrier such as Aquaphor®. The active ingredients consist of a metallic composition made up of 15.0 g of chromium oxide and 15.0 g of iron compound. The iron compound is made up of 7.5 g of iron/sulfur oxide and 7.5 g of iron/chloride oxide. The active ingredients make up 5% of the total weight of the treatment substance, and the carrier makes up 95% of the total weight of the treatment substance. The weight of chromium oxide and the weight of iron compound are each 50% of the total weight of the active ingredients. The iron compound by weight is 50% iron/sulfur oxide and 50% iron/chloride oxide. Therefore, the weight of iron/sulfur oxide is 25% the total weight of the active ingredients, and the weight of iron/chloride oxide is 25% the total weight of the active ingredients. The weight of chromium oxide is 2.5% the total weight of the treatment substance, and the total weight of the iron compound is 2.5% the total weight of the treatment substance. The weight of iron/sulfur oxide and the weight of iron/chloride oxide are each 1.25% the total weight of the treatment substance.

### EXAMPLE 2

120 g of a treatment substance is made from 6 g of active ingredients in 114 g of a carrier such as Aquaphor®. The active ingredients consist of 5.0 g of a metallic composition and 1.0 g of a time release compound. The metallic composition is composed of 2.5 g of chromium oxide and 2.5 g of iron compound. The iron compound consists entirely of iron oxide. The active ingredients make up 5% of the total weight of the treatment substance and the carrier makes up 95% of the total weight of the treatment substance. The weight of chromium oxide and the weight of iron compound are each 41.67% of the total weight of the active ingredients. The weight of the time release compound is 16.66% the total weight of the active ingredients. The time release compound is a hydrogen dioxide (H₂O₂) time release cream. The weight of chromium oxide is 2.08% of the total weight of the treatment substance. The weight of iron compound is also 2.08% of the total weight of the treatment substance. The weight of the time release compound is 0.84% of the total weight of the treatment substance.

### EXAMPLE 3

240 g of a treatment substance is made from 12 g of active ingredients in 228 g of a carrier such as Aquaphor®. The active ingredients consist of a metallic composition made up of 6 g of chromium oxide and 6 g of iron compound. The iron compound consists in its entirety of iron/sulfur oxide. The active ingredients make up 5% of the total weight of the treatment substance and the carrier makes up 95% of the total weight of the treatment substance. The weight of chromium oxide and the weight of the iron compound are each 50% of the total weight of the active ingredients. The weight of chromium oxide and the weight of the iron compound are each 2.5% of the total weight of the treatment substance.

### EXAMPLE 4

240 g of a treatment substance is made from 12 g of active ingredients in 228 g of a carrier such as Aquaphor®. The active ingredients consist of a metallic composition made up of 7.2 g of chromium oxide and 4.8 g of iron compound. The iron compound consists entirely of iron/sulfur oxide. The active ingredients make up 5% of the total weight of the treatment substance and the carrier makes up 95% of the total weight of the treatment substance. The weight of chromium oxide is 60% the total weight of the active ingredients and 3% of the total weight of the treatment substance. The weight of iron compound is 40% the total weight of the active ingredients and 2% of the total weight of the treatment substance.

### EXAMPLE 5

150 g of a treatment substance is made from 7.5 g of active ingredients in 142.5 g of a carrier such as Aquaphor®. The active ingredients are a metallic composition consisting of 3.0 g of chromium oxide and 4.5 g of iron compound. The iron compound is made up of 3.0 g of iron/sulfur oxide and 1.5 g of iron chloride. The active ingredients make up 5% of the total weight of the treatment substance and the carrier makes up 95% of the total weight of the treatment substance. The weight of chromium oxide is 40% of the total weight of the active ingredients. The iron compound by weight is 60% of the total weight of the active ingredients. The iron/sulfur oxide is 40% by weight of the total weight of the active ingredients, and the weight of iron chloride is 20% of the total weight of the active ingredients. The iron/sulfur oxide by weight is two-thirds the total weight of the iron compound, and the iron chloride by weight is one third the total weight of the iron compound. The weight of chromium oxide is 2% of the total weight of the treatment substance. The weight of iron compound is 3% of the total weight of the treatment substance. The weight of iron/sulfur oxide accounts for 2% total weight of the treatment substance while the weight of iron chloride accounts for 1% of the total weight of the treatment substance.

### EXAMPLE 6

147.6 g of treatment substance is made from 7.38 g of active ingredients in 140.22 g of a carrier such as Aquaphor®. The active ingredients consist of 6.0 g of metallic composition and 1.38 g of time release compound. The metallic composition consists of 3.0 g of chromium oxide and 3.0 g of iron compound. The iron compound consists in its entirety of iron/sulfur oxide. The active ingredients make up 5% of the total weight of the treatment substance and the carrier makes up 95% of the total weight of the treatment substance. The weight of chromium oxide and the weight of the iron compound are each 40.65% of the total weight of the active ingredients. The time release compound by weight is 18.70% of the total weight of the active ingredients. The weight of chromium oxide is 2.03% the total weight of the treatment substance, and the weight of iron compound is also 2.03% the total weight of the treatment substance. The weight of time release compound is 0.94% the total weight of the treatment substance. The time release compound is hydrogen dioxide (H₂O₂) suspended release gel.

### EXAMPLE 7

34 g of treatment substance is made from 1.7 g of active ingredients in 32.3 g of a carrier. The active ingredients are a metallic composition consisting of 0.75 g of chromium oxide and 0.95 g of iron compound. The iron compound consists of 0.75 g of iron/sulfur oxide and 0.20 g of iron/chloride oxide. The active ingredients make up 5% of the total weight of the treatment substance and the carrier makes up 95% of the total weight of the treatment substance. The weight of chromium oxide is 44% of the total weight of the active ingredients. The weight of iron compound is 56% of the total weight of the active ingredients. The weight of iron/sulfur oxide is 44% of the total weight of the active ingredients while the weight of iron/chloride oxide is 12% of the total weight of the active ingredients. The weight of iron/sulfur oxide is 79% of the total weight of the iron compound while the weight of iron/chloride oxide accounts for 21% of the total weight of the iron compound. The weight of chromium oxide and the weight of the iron/sulfur oxide are each 2.2% of the total weight of the treatment substance. The weight of the iron/chloride oxide is 0.6% the total weight of the treatment substance. The weight of the iron compound thusly accounts for 2.8% of the total weight of the treatment substance. The carrier consists of VanPen cream with a color modifier. In particular, the carrier consists of 24.871 g of VanPen cream and 7.429 g of titanium dioxide as the color modifier. Accordingly, the weight of VanPen cream accounts for 77% of the total weight of the carrier while the weight of the color modifier accounts for 23% of the total weight of the carrier. The VanPen cream by weight is 73.15% of the total weight of the treatment substance, and the color modifier by weight is 21.85% of the total weight of the treatment substance.

In a method of topical treatment according to the present invention, a treatment substance as described above is applied to the site of an actual or potential abnormal biological condition on an anatomical surface of a body. The treatment substance may be applied by spreading a layer of the treatment substance on the anatomical surface. The treatment substance may be gently rubbed into the anatomical surface and/or is allowed to be absorbed by the anatomical surface. Application of the treatment substance may be self-performed by the patient or user. The treatment substance, in the presence of oxygen, creates an oxidative chemical reaction capable of treating the actual or potential abnormal biological condition. Treatment includes normalizing the actual or existing abnormal biological condition or preventing the potential or not yet existing abnormal biological condition. A chemical reaction can be produced capable of destroying bacteria, fungi and viruses. Additionally, the reaction can be produced to have an anti-inflammatory effect. The treatment substance is capable of treating various symptomatic and asymptomatic abnormal biological conditions including abnormal fungal, bacterial, biofllm, viral, inflammatory and neoplastic biological conditions. The treatment substance is capable of preventing, reducing or eliminating biofilm. The abnormal biological conditions treated by the present invention may be due to internal causes such as disease and immunity deficiencies or external causes such as injuries, trauma, burns and insect bites. Treatment according to the present invention may be primary or preventative. The treatment substance may be applied to the site of the actual or potential abnormal biological condition on the anatomical surface of the body at regular intervals. Typically, a complete course of treatment will involve multiple applications of the treatment substance at regular or repeated intervals over time.

Inasmuch as the present invention is subject to many variations, modifications and changes in detail, it is intended that all subject matter discussed above and set forth in the accompanying examples be interpreted as illustrative only and not be taken in a limiting sense.

1. A topical treatment for abnormal biological conditions occurring on an anatomical surface of a body, comprising
a treatment substance comprising chromium oxide, an iron compound and a pharmaceutically acceptable carrier in a consistency for topical application to the anatomical surface of the body.

2. A topical treatment as recited in claim 1 wherein said iron compound consists of one or more of iron/sulfur oxide, iron/chloride oxide, iron chloride or iron oxide.

3. A topical treatment as recited in claim 2 wherein said iron compound consists entirely of iron oxide or iron/sulfur oxide.

4. A topical treatment as recited in claim 2 wherein said iron compound consists of iron/sulfur oxide and iron/chloride oxide or iron/chloride.

5. A topical treatment as recited in claim 2 wherein said treatment substance has a total weight, said chromium oxide has a weight in the range of about 2-3% of said total weight of said treatment substance and said iron compound has a weight in the range of about 2-3% of said total weight of said treatment substance.

6. A topical treatment as recited in claim 2 wherein said treatment substance further comprises a time release compound.

7. A treatment substance as recited in claim 2 wherein said carrier comprises Aquaphor®, Hydrophor or VanPen.

8. A topical treatment for abnormal biological conditions occurring on an anatomical surface of a body, comprising
a treatment substance comprising active ingredients in a pharmaceutically acceptable carrier in a consistency for topical application to the anatomical surface of the body, said active ingredients including chromium oxide and an iron compound, said iron compound including one or more of iron oxide, iron/sulfur oxide, iron/chloride oxide or iron chloride, said treatment substance having a total weight, said active ingredients having a total weight of about 5% of said total weight of said treatment substance, said carrier having a total weight of about 95% of said total weight of said treatment substance, said chromium oxide having a weight in the range of about 40-60% of said total weight of said active ingredients, and said iron compound having a total weight in the range of about 40-60% of said total weight of said active ingredients.

9. A treatment substance as recited in claim 8 wherein said iron compound consists entirely of iron oxide or iron/sulfur oxide.

10. A treatment substance as recited in claim 8 wherein said iron compound consists of iron/sulfur oxide and either iron/chloride oxide or iron chloride.

11. A treatment substance as recited in claim 10 wherein said iron/sulfur oxide has a weight at least 50% of said total weight of said iron compound.

12. A treatment substance as recited in claim 11 wherein said iron compound consists of said iron/sulfur oxide and said iron/chloride oxide, said iron/chloride oxide having a weight in the range of 20 to 50% of said total weight of said iron compound.

13. A treatment substance as recited in claim 12 wherein said weight of said iron/sulfur oxide is about 80% of said total weight of said iron compound and said weight of said iron/chloride oxide is about 20% of said total weight of said iron compound.

14. A treatment substance as recited in claim 11 wherein said iron compound consists of said iron/sulfur oxide and said iron chloride, said weight of said iron/sulfur oxide being about two thirds said total weight of said iron compound and said iron chloride having a weight about one third said total weight of said iron compound.

15. A treatment substance as recited in claim 8 wherein said active ingredients further comprise a time release compound.

16. A treatment substance as recited in claim 15 wherein said time release compound has a weight in the range of about 15-20% said total weight of said active ingredients.

17. A treatment substance as recited in claim 8 wherein said carrier comprises Aquaphor®, Hydraphor or VanPen.

18. A treatment substance as recited in claim 17 wherein said carrier consists of VanPen and a color modifier.

19. A treatment substance as recited in claim 18 wherein said carrier has a total weight and said color modifier is titanium dioxide having a weight about 23% of said total weight of said carrier.

20. A method of treating an abnormal biological condition occurring on an anatomical surface of a body, comprising the steps of
applying a treatment substance comprising chromium oxide, an iron compound and a pharmaceutically acceptable carrier to the site of an actual or potential abnormal biological condition on the anatomical surface of the body; and
repeating said step of applying at regular intervals to treat the abnormal biological condition.
